(19) United States
(12) Patent Application Publication
(10) Pub. No.: US 2022/0016142 A1
(43) Pub. Date: Jan. 20, 2022

(54) COMBINATION THERAPY FOR TREATMENT OF CANCER

(71) Applicant: PLUS VITECH, S.L., Sevilla (ES)

(72) Inventors: Francisco José LÓPEZ-HERNÁNDEZ, Sevilla (ES); Felipe VOCES-SÁNCHEZ, Sevilla (ES); Josep LAMARCA-CIURO, Sevilla (ES); Yaremi QUIRÓS-LUIS, Sevilla (ES); Anna ESTEVE-ARENYS, Sevilla (ES); Laura HIRALDO-GONZÁLEZ, Sevilla (ES); Manuel Vicente SALINAS-MARTÍN, Sevilla (ES)

(73) Assignee: PLUS VITECH, S.L., Sevilla (ES)

(21) Appl. No.: 17/414,849

(22) PCT Filed: Dec. 20, 2019
(86) PCT No.: PCT/EP2019/086817

§ 371 Date: Jun. 16, 2021

(30) Foreign Application Priority Data
Dec. 21, 2018 (EP) 18382975.3

(51) Int. Cl. A61K 31/675; A61P 35/00; A61K 31/496; A61K 31/4375; A61K 31/5377; A61K 39/395

(52) U.S. Cl. CPC A61K 31/675; A61P 35/00; A61K 31/496; A61K 2039/505; A61K 31/5377; A61K 39/3955; A61K 31/4375

(57) ABSTRACT

The present invention relates to the treatment of cancer using a combination of (a) a NK inhibitor (b) a checkpoint inhibitor.

### FIELD OF THE INVENTION

The present invention relates to the treatment of cancer using a combination of (a) a NK₁ inhibitor, which is preferably aprepitant or fosaprepitant, or pharmaceutically acceptable salt thereof, and (b) a checkpoint inhibitor, such as a PD-1 antagonist.

### BACKGROUND TO THE INVENTION

Programmed cell death protein 1 (PD-1) is a cell surface receptor that plays an important role in down-regulating the immune system. This down regulation of the immune system prevents autoimmune diseases, but also can prevent the immune system from killing cancer cells. Drugs that block PD-1 can therefore activate the immune system, which can then attack cancer cells. PD-1 antagonists can therefore provide a useful therapeutic approach for treating cancer.

Nivolumab and pembrolizumab target PD-1, and have been approved for treatment of a variety of different cancers, including melanoma and non-small cell lung cancer. There are many other compounds under development that target PD-1, including pidilizumab.

Although blocking PD-1 provides a useful therapeutic approach for treating many cancers, the stimulation of the immune system can cause unwanted immune-related adverse reactions in patients. These unwanted immune-related adverse reactions can reduce the desirability of treating patients, particularly those prone to immune-related adverse reactions and/or already suffering from autoimmune diseases, with drugs that target PD-1.

In addition to targeting PD-1, a number of other treatments have emerged which target other immune system checkpoints, such as CTLA-4, and are collectively known as “checkpoint inhibitor”. However, many of these treatments suffer from the same limitations as discussed above for PD-1 blockers.

Aprepitant and its prodrug fosaprepitant are neurokinin 1 (NK₁) inhibitors that have been approved for treating nausea and vomiting, for example acute or delayed chemotherapy-induced nausea and vomiting, or post-operative nausea and vomiting. Aprepitant has also been investigated for use in treating a variety of other diseases, including depression and cancer. The latter is discussed, for example, in EP 2 837 381 A1. Other NK₁ inhibitors are well known to those skilled in the art.

Despite these therapeutic advances, there remains an urgent need to develop more effective, well-tolerated and affordable treatments to improve symptoms and survival rates for sufferers of cancers.

### SUMMARY OF THE INVENTION

It has now been found that a combination therapy of a checkpoint inhibitor (such as a PD-1 antagonist) with a NK₁ inhibitor (such as aprepitant or fosaprepitant, or a pharmaceutically acceptable salt thereof), results in a surprising improvement in therapeutic efficacy in treating cancers as compared to the corresponding monotherapies. The enhanced therapeutic effect that is observed with the combination therapy is highly advantageous from a clinical perspective.

A particularly desirable advantage associated with the combination therapy is that each component of the combination can be used at lower doses than would typically be used for a corresponding monotherapy, without reducing the clinical efficacy of the treatment. The reduction in the dose of each component required to achieve clinical efficacy means that there is an associated reduction in side effects observed. This reduction in side effects is highly desirable.

The present invention thus provides a pharmaceutical composition which comprises: (a) a NK₁ inhibitor, which is preferably aprepitant or fosaprepitant, or pharmaceutically acceptable salt thereof, and (b) a checkpoint inhibitor, such as a PD-1 antagonist, for use in treating cancer.

The present invention further comprises:
a NK₁ inhibitor, which is preferably aprepitant or fosaprepitant, or pharmaceutically acceptable salt thereof, for use in treating cancer by co-administration with a checkpoint inhibitor, such as a PD-1 antagonist;
a checkpoint inhibitor, such as a PD-1 inhibitor, for use in treating cancer, by co-administration with a NK₁ inhibitor a NK₁ inhibitor, which is preferably aprepitant or fosaprepitant, or pharmaceutically acceptable salt thereof;
a method of treating a patient suffering from cancer, which method comprises co-administering to said patient (a) a NK₁ inhibitor, which is preferably aprepitant or fosaprepitant, or pharmaceutically acceptable salt thereof, and (b) a checkpoint inhibitor, such as a PD-1 antagonist;
a product comprising (a) a NK₁ inhibitor, which is preferably aprepitant or fosaprepitant, or pharmaceutically acceptable salt thereof, and (b) a checkpoint inhibitor, such as a PD-1 antagonist, as a combined preparation for simultaneous, concurrent, separate or sequential use in the treatment of a patient suffering from cancer;
use of a NK₁ inhibitor, which is preferably aprepitant or fosaprepitant, or pharmaceutically acceptable salt thereof, in the manufacture of a medicament for the treatment of cancer by co-administration with a checkpoint inhibitor, such as a PD-1 antagonist;
use of a checkpoint inhibitor, such as a PD-1 antagonist, in the manufacture of a medicament for the treatment of cancer by co-administration with a NK₁ inhibitor, which is preferably aprepitant or fosaprepitant, or pharmaceutically acceptable salt thereof;
a pharmaceutical composition which comprises:
(a) a NK₁ inhibitor, which preferably is aprepitant or fosaprepitant, or pharmaceutically acceptable salt thereof, and
(b) a checkpoint inhibitor, such as a PD-1 antagonist; and kit which comprises:
(a) a pharmaceutical composition comprising a NK₁ inhibitor, which is preferably aprepitant or fosaprepitant, or pharmaceutically acceptable salt thereof; and
(b) a pharmaceutical composition comprising a checkpoint inhibitor, such as a PD-1 antagonist.

### BRIEF DESCRIPTION OF THE FIGURES

FIG. 1 shows the results from the first experiment in Example 1, in which mice were treated with fosaprepitant and the evolution of tumour volume was measured. The mice were treated with 0 (control), 3, 10, 30 or 60 mg/kg fosaprepitant (FosAPT). The black arrow shows the day when treatments started.

FIG. 2 shows the results from the second experiment in Example 1, in which mice were treated with an antibody antagonist of PD-1 and the evolution of tumour volume was measured. The mice were treated with 0 (Control), 1, 3 and 10 mg/kg anti-PD-1 antibody. The black arrow shows the day when treatments started, with treatments being administered on alternate days.

FIG. 3 shows the results from the third experiment in Example 1, in which mice received a series of different treatments [fosaprepitant (30 mg/kg/day), antibody antagonist of PD-1 (8 mg/kg on alternate days) or isotype gammaglobulin (8 mg/kg on alternate days), and combinations thereof as detailed in Example 1. The evolution of tumour volume was measured. Statistical analysis was performed with the Kruskal-Wallis test. * Z>1.96 vs Control, Isotype and Anti-PD1. The black arrow shows the day when treatments started.

FIG. 4 shows the results from Example 2a. The arrow represents the first day of treatment (day 4 after cell inoculation). All of the tested treatments failed to inhibit tumour growth of pulmonary carcinoma tumours.

FIG. 5 shows the results from Example 2b. The arrow represents the first day of treatment (day 8 after cells inoculation). Maropitant and CTLA-4 each individually inhibited the grown of melanoma tumours, with the combination exhibiting a great than additive effect.

### DETAILED DESCRIPTION

### NK₁ Inhibitors

NK₁ inhibitors are a well-known class of drug, and any suitable NK₁ inhibitor can be used in the present invention.

Typically, the NK₁ inhibitor is aprepitant, fosaprepitant, netupitant, maropitant, vestipitant, casopitant, vofopitant, ezlopitant, lanepitant, LY-686017, L-733,060, L-732,138, L-703,606, WIN 62,577, CP-122721, TAK-637, R673, CP-100263, WIN 51708, CP-96345, L-760 735, CP-122721, L-758 298, L-741 671, L-742 694, CP-99994 or T-2328, or a pharmaceutically acceptable salt of any thereof.

Preferably, the NK₁ inhibitor is aprepitant, fosaprepitant, netupitant, maropitant, vestipitant, casopitant, vofopitant, ezlopitant or lanepitant, or a pharmaceutically acceptable salt of any thereof.

More preferably, the NK₁ inhibitor is aprepitant, fosaprepitant or netupitant, maropitant, or a pharmaceutically acceptable salt of any thereof.

Most preferably, the NK₁ inhibitor is aprepitant or its prodrug fosaprepitant, or a pharmaceutically acceptable salt of either thereof.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines such as meglumine, aralkyl amines or heterocyclic amines.

Aprepitant has the following structure:

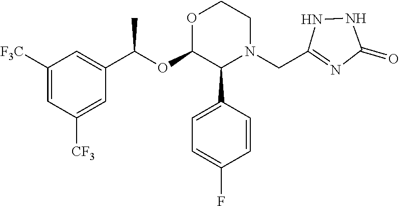 [SMILES: C[C@@H](O[C@H]1OCCN(Cc2nc(=O)[nH][nH]2)[C@H]1c1ccc(F)cc1)c1cc(C(F)(F)F)cc(C(F)(F)F)c1]

Aprepitant is not typically formulated in the form of a pharmaceutically acceptable salt. Thus, in a preferred aspect of the invention the NK₁ inhibitor is aprepitant.

Fosaprepitant is prodrug of aprepitant and has the following structure:

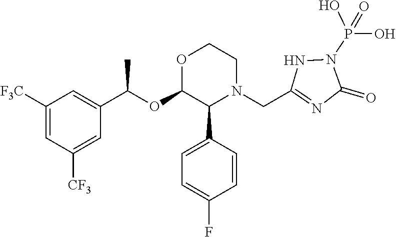 [SMILES: C[C@@H](O[C@H]1OCCN(Cc2nc(=O)n(P(=O)(O)O)[nH]2)[C@H]1c1ccc(F)cc1)c1cc(C(F)(F)F)cc(C(F)(F)F)c1]

Fosaprepitant is typically provided in the form of a pharmaceutically acceptable salt, preferably in the form of the dimeglumine salt:

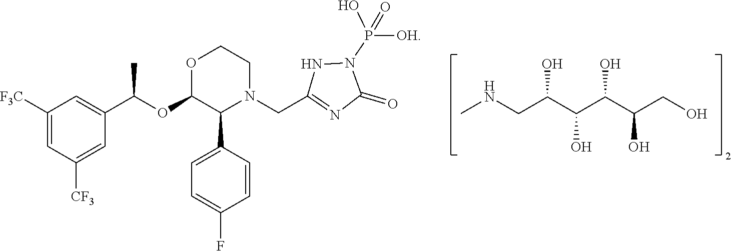

Thus, in a preferred aspect of the invention, the NK₁ inhibitor is fosaprepitant dimeglumine.

Pharmaceutically acceptable salts of fosaprepitant, such as fosaprepitant dimeglumine, are typically reconstituted in an aqueous solvent, such as saline, prior to administration, thereby providing an aqueous solution comprising fosaprepitant.

Fosaprepitant is converted in vivo to aprepitant. Thus, when administered to a patient, typically intravenously, fosaprepitant is converted to aprepitant.

### Checkpoint Inhibitors

A checkpoint point inhibitor used herein to refer to an agent which, when administered to a subject, blocks or inhibits the action of an immune system checkpoint resulting in the upregulation of an immune effector response in the subject, typically a T cell effector response, which preferably comprises an anti-tumour T cell effector response. The checkpoint inhibitor may therefore block or inhibit any of the immune system checkpoints described below. The agent may be an antibody or any other suitable agent which results in said blocking or inhibition. Other suitable inhibitors include small molecule inhibitors (SMI), which are typically small organic molecules.

An “antibody” as used herein includes whole antibodies and any antigen binding fragment (i.e., “antigen-binding portion”) or single chains thereof. An antibody may be a polyclonal antibody or a monoclonal antibody and may be produced by any suitable method. Examples of binding fragments encompassed within the term “antigen-binding portion” of an antibody include a Fab fragment, a F(ab′)2 fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody.

Effector T cell activation is normally triggered by the T cell receptor recognising antigenic peptide presented by the MHC complex. The type and level of activation achieved is then determined by the balance between signals which stimulate and signals which inhibit the effector T cell response. The term “immune system checkpoint” is used herein to refer to any molecular interaction which alters the balance in favour of inhibition of the effector T cell response. That is, a molecular interaction which, when it occurs, negatively regulates the activation of an effector T cell. Such an interaction might be direct, such as the interaction between a ligand and a cell surface receptor which transmits an inhibitory signal into an effector T cell. Or it might be indirect, such as the blocking or inhibition of an interaction between a ligand and a cell surface receptor which would otherwise transmit an activatory signal into the effector T cell, or an interaction which promotes the upregulation of an inhibitory molecule or cell, or the depletion by an enzyme of a metabolite required by the effector T cell, or any combination thereof.

Examples of immune system checkpoints include:
a) The interaction between PD-1 and PDL-1 and/or PD-1 and PDL-2;
b) The interaction between CTLA-4 and CD86 and/or CTLA-4 and CD80;
c) The interaction between B7-H3 and/or B7-H4 and their respective ligands;
d) The interaction between HVEM and BTLA;
e) The interaction between GALS and TIM3;
f) The interaction between MHC class I or II and LAG3; and
g) The interaction between MHC class I or II and KIR

A preferred checkpoint for the purposes of the present invention is checkpoint (a), namely the interaction between PD-1 and either of its ligands PD-L1 and PD-L2. PD-1 is expressed on effector T cells. Engagement with either ligand results in a signal which downregulates activation. The ligands are expressed by some tumours. PD-L1 in particular is expressed by many solid tumours, including melanoma. These tumours may therefore down regulate immune mediated anti-tumour effects through activation of the inhibitory PD-1 receptors on T cells. By blocking the interaction between PD-1 and one or both of its ligands, a checkpoint of the immune response may be removed, leading to augmented anti-tumour T cell responses. Therefore PD-1 and its ligands are examples of components of an immune system checkpoint which may preferably be targeted in the invention

Another preferred checkpoint for the purposes of the present invention is checkpoint (b), namely the interaction between the T cell receptor CTLA-4 and its ligands, the B7 proteins (B7-1 and B7-2). CTLA-4 is ordinarily upregulated on the T cell surface following initial activation, and ligand binding results in a signal which inhibits further/continued activation. CTLA-4 competes for binding to the B7 proteins with the receptor CD28, which is also expressed on the T cell surface but which upregulates activation. Thus, by blocking the CTLA-4 interaction with the B7 proteins, but not the CD28 interaction with the B7 proteins, one of the normal check points of the immune response may be removed, leading to augmented anti-tumour T cell responses. Therefore CTLA4 and its ligands are examples of components of an immune system checkpoint which may preferably be targeted in the invention

### PD-1 Antagonists

A preferred example of a checkpoint inhibitor useful in the present invention is a PD-1 antagonist. A PD-1 antagonist is used herein to refer to any substance which inhibits the activity of the PD-1 signalling pathway. A PD-1 antagonists may achieve an inhibitory effect by any mechanism. For example, it may directly interfere with downstream signalling from an active PD-1 molecule, or it may reduce or block the interaction between PD-1 and its ligands, thereby limiting or preventing the activation of PD-1. Accordingly a PD-1 antagonist may bind directly to PD-1 or to one of its ligands, such as PD-L1 or PD-L2. A PD-1 antagonist may be a small molecule inhibitor (SMI; typically a small organic molecule) but is preferably an antibody. The antibody may be specific for PD-1, PD-L1 or PD-L2, but is preferably specific for PD-1.

Preferably the PD-1 antagonist is nivolumab, pembrolizumab or pidilizumab. Nivolumab and pembrolizumab are particularly preferred.

In a preferred aspect of the invention, the PD-1 antagonist is nivolumab.

In a further preferred aspect of the invention, the PD-1 antagonist is pembrolizumab.

In a further preferred aspect of the invention, the PD-1 antagonist is pidilizumab.

### CTLA-4 Antagonists

A preferred example of a checkpoint inhibitor useful in the present invention is a CTLA-4 antagonist. A CTLA-4 antagonist is used herein to refer to any substance which inhibits the activity of the CTLA-4 signalling pathway. A CTLA-4 antagonist may achieve an inhibitory effect by any mechanism. For example, it may directly interfere with downstream signalling from an active CTLA-4 molecule, or it may reduce or block the interaction between CTLA-4 and its ligands, thereby limiting or preventing the activation of CTLA-4. Accordingly a CTLA-4 antagonist may bind directly to CTLA-4 or to one of its ligands, such as the B7 proteins (B7-1 and B7-2). A CTLA-4 antagonist may be a small molecule inhibitor (SMI; typically a small organic molecule) but is preferably an antibody.

Preferably, the CTLA-4 antagonist is ipilumumab or tremelimumab.

### Treatment of Cancer

Typically the patient to be treated is a mammal. Preferably the patient is a human.

Treatment may be curative or palliative in nature, i.e. it may aim at curing the patient, achieving complete or partial remission, alleviating or managing symptoms and/or side effects of the disease (without curing the patient) and/or increasing life expectancy.

The treatment may be an adjuvant therapy or a neo-adjuvant therapy, for example being combined with surgery, other chemotherapy strategies and/or radiotherapy. Alternatively, the treatment may be carried out in the absence of surgery, other chemotherapy strategies and/or radiotherapy.

The cancer to be treated may be metastatic or non-metastatic and may be resectable or unresectable. The cancer may also be refractory to conventional chemotherapies.

The cancer to be treated is typically melanoma, lung cancer, renal cell carcinoma, lymphoma, squamous cell cancer, or urothelial carcinoma. The cancer to be treated is preferably melanoma, renal cell carcinoma, lymphoma, squamous cell cancer, or urothelial carcinoma, preferably melanoma.

The melanoma is typically advanced (unresectable or metastatic) melanoma.

The lung cancer is typically non-small cell lung cancer, preferably locally advanced or metastatic non-small cell lung cancer. The non-small cell lung cancer may be refractory to earlier chemotherapy.

However, in an alternative aspect of the invention, it is preferred that the cancer to be treated is not lung cancer.

The renal cell carcinoma is typically advanced renal cell carcinoma. The renal cell carcinoma may be refractory to earlier chemotherapy.

The lymphoma is typically classical Hodgkin lymphoma, preferably relapsed or refractory classical Hodgkin lymphoma. The classical Hodgkin lymphoma may have been earlier treated by autologous stem cell transplant (ASCT) and treatment with brentuximab vedotin. Typically such an earlier treatment has failed. Alternatively, the patient with classical Hodgkin lymphoma may be ineligible for ASCT and/or have failed brentuximab vedotin treatment.

The squamous cell cancer is typically squamous cell cancer of the head and neck. The squamous cell cancer of the head and neck may have earlier been treated with, or is simultaneously being treated with, a platinum-based therapy such as cis-platin.

The urothelial carcinoma is typically locally advanced or metastastic urothelial carcinoma. The urothelial carcinoma may be refractory to platinum-based therapies such as cis-platin.

It is particularly preferred that cancer is one in which PD-1 or PDL-1 is expressed in the tumour. A tumour in which PD-1 or PDL-1 is expressed is preferably one in which more than 1% of the cells of the tumour express PD-1 or PDL-1 when assessed using any suitable technique. Suitable techniques include immunohistochemistry.

It is also particularly preferred that the cancer shares a molecular profile with melanoma. This similarity of the molecular profile of the cancer to the molecular profile of melanoma can be determined by assessing one or more of the following biomarkers:
a) the level of expression of NK1 receptor, or the mRNA that encodes it, and/or
b) the presence of mutation in the K-ras gene, and/or
c) the level of expression of ERK proteins, preferably ERK1 and ERK2, or the mRNA encoding the same, and/or
d) the level of expression of MEK proteins, preferably MEK1 and MEK2, or the mRNA encoding the same, and/or
e) the level of expression of the AKT protein, preferably AKT1 and AKT2, or the mRNA encoding the same, and/or
f) the presence or not of the truncated form of the NK1 receptor, or the mRNA encoding the same, and/or
g) the presence of the NK1 receptor constituently activated,
   These biomarkers can be compared to the values observed in melanoma.

For example, if one or more of a) the level of expression of NK1 receptor, or the mRNA that encodes it, c) the level of expression of ERK proteins, preferably ERK1 and ERK2, or the mRNA encoding the same, d) the level of expression of MEK proteins, preferably MEK1 and MEK2, or the mRNA encoding the same, and e) the level of expression of the AKT protein, preferably AKT1 and AKT2, or the mRNA encoding the same, are ±20% (for example ±10%) the value observed in melanoma, then the cancer is considered to have a similar molecular profile to melanoma.

Similarly, if b) mutation in the K-ras gene is absent, and/or f) the presence of the truncated form of the NK1 receptor, or the mRNA encoding the same, is detected, and/or g) the presence of the NK1 receptor constituently activated is detected, then the cancer is considered to have a similar molecular profile to melanoma.

The measurement of biomarkers a) to g) is described in detail in WO 2014/122353, the content of which is hereby incorporated by reference. Thus, a skilled person can assess each of these biomarkers, both in melanoma and in the cancer being assessed, and thereby determine without difficulty whether the cancer has a similar molecular profile to melanoma.

### Pharmaceutical Compositions

The present invention involves the use of a combination of (a) a NK₁ inhibitor, which is preferably aprepitant or fosaprepitant, or a pharmaceutically acceptable salt thereof, and (b) a checkpoint inhibitor, such as a PD-1 antagonist. The NK₁ inhibitor and checkpoint inhibitor are herein referred to as “active ingredients”.

In one aspect, the present invention provides a pharmaceutical composition that comprises: (a) a NK₁ inhibitor, which is preferably aprepitant or fosaprepitant, or a pharmaceutically acceptable salt thereof, and (b) checkpoint inhibitor, such as a PD-1 antagonist; including for use in treating cancer. Pharmaceutical compositions according to the invention will typically further comprise one or more pharmaceutically acceptable excipients or carriers.

The present invention extends to situations where the active ingredients discussed above are co-administered. When the active ingredients are co-administered they can be present either in a single pharmaceutical composition or in separate pharmaceutical compositions, including in separate pharmaceutical compositions optimized for administration either by the same mode or a different mode. For example, the active ingredients may both be administered intravenously, either in a single pharmaceutical composition or, more preferably, in separate pharmaceutical compositions.

For the avoidance of doubt, in the product comprising (a) a NK₁ inhibitor, which is preferably aprepitant or fosaprepitant, or a pharmaceutically acceptable salt thereof, and (b) a checkpoint inhibitor, such as a PD-1 antagonist, as a combined preparation for simultaneous, concurrent, separate or sequential use, the product may comprise either a single pharmaceutical composition that comprises both (a) and (b) (i.e. a unit dosage form) or alternatively, and preferably, a first pharmaceutical composition that comprises (a) and a second (i.e., separate) pharmaceutical composition that comprises (b).

Co-administration of the active ingredients according to the present invention includes simultaneous, separate and sequential administration.

In general, administration of the pharmaceutical compositions may be oral (as syrups, tablets, capsules, lozenges, controlled-release preparations, fast-dissolving preparations, etc), by injection (subcutaneous, intradermal, intramuscular, intravenous, etc.), or by inhalation (as a dry powder, a solution, a dispersion, etc.).

The preferred route of administration will depend upon the specific active ingredient to be delivered, and a skilled person can easily choose an appropriate route. For example, aprepitant is preferably delivered orally, whereas fosaprepitant is preferably administered intravenously. If the checkpoint inhibitor, such as a PD-1 antagonist, is an antibody, it is typically administered as a systemic infusion, for example intravenously. If the checkpoint inhibitor, such as a PD-1 antagonist, is an SMI it is typically administered orally.

For oral administration, the pharmaceutical compositions of the present invention may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

For administration by injection, the pharmaceutical compositions typically take the form of an aqueous injectable solution. Examples of suitable aqueous carriers that may be employed in the injectable pharmaceutical compositions of the invention include water, buffered water and saline. In many cases, it will be preferable to include isotonic agents, for example, sugars, polyalcohols such as mannitol, sorbitol, or sodium chloride in the composition.

For administration by inhalation, the pharmaceutical composition may take the form of a dry powder, which will typically comprise the active ingredient and a carrier such as lactose, and be delivered via an inhaler. Alternatively, the pharmaceutical composition may for example be formulated as aqueous solutions or suspensions and be delivered as an aerosol from a pressurised metered dose inhaler, with the use of a suitable liquefied propellant. Suitable propellants include fluorocarbon or hydrogen-containing chlorofluorocarbon or mixtures thereof, particularly hydrofluoroalkanes.

Pharmaceutical compositions comprising of the invention may be prepared by any suitable method known to those of skill in the art.

Pharmaceutical compositions of the invention may comprise additional active ingredients, such as an additional therapeutic or prophylactic agent intended, for example, for the treatment of the same condition or a different one, or for other purposes such as amelioration of side effects. However, it is generally preferred that the compositions of the invention do not contain any further active ingredients (i.e. the pharmaceutical compositions contain only (a) a NK₁ inhibitor, which is preferably aprepitant or fosaprepitant, or a pharmaceutically acceptable salt thereof, and (b) a checkpoint inhibitor, such as a PD-1 antagonist, as active ingredients).

### Dosages and Dosage Regimes

Suitable dosages of the active ingredients used in the present invention may easily be determined by a skilled medical practitioner.

Actual dosage levels of the active ingredients in the pharmaceutical compositions of the present invention may be varied so as to obtain an amount of the active ingredient, which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Appropriate doses for antibodies and SMIs may be determined by a physician. Appropriate doses for antibodies are typically proportionate to the body weight of the subject. A suitable dosage of an antibody may be determined by a skilled medical practitioner. Actual dosage levels of an antibody may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antibody employed, the route of administration, the time of administration, the rate of excretion of the antibody, the duration of the treatment, other drugs, compounds and/or materials used in combination with the particular compositions employed, the age, sex, weight, condition, general health and prior medical history of the patient being treated, and like factors well known to the skilled person. A suitable dose of an antibody may be, for example, in the range of from about 0.1 μg/kg to about 100 mg/kg body weight of the patient to be treated. For example, a suitable dosage may be from about 1 μg/kg to about 10 mg/kg body weight per day or from about 10 μg/kg to about 5 mg/kg body weight per day.

It is a finding of the present invention that a profound anti-cancer effect may be observed even when the (a) a NK₁ inhibitor, and/or (b) a checkpoint inhibitor, such as a PD-1 antagonist, are administered to the patient at a low dose which provides little or no therapeutic effect in treating cancer when administered alone. Thus, the dosage of (a) a NK₁ inhibitor, and/or (b) a checkpoint inhibitor, such as a PD-1 antagonist, is typically a low, or sub-clinical, dosage. The use of low, or sub-clinical, dosages has significant advantages in reducing the side effects observed in patients. For example, as discussed above, checkpoint inhibitor, such as a PD-1 antagonists, can give rise to immune-related adverse reactions due to their stimulating effect on the immune system.

By administering the checkpoint inhibitor, such as a PD-1 antagonist, at a lower dose than usual, the risk of immune-related adverse reactions can be reduced. This means that the compositions of the invention can be used to treat patients prone to or who have previously experienced immune-related adverse reactions and/or patients already suffering from autoimmune diseases. Thus, in a preferred aspect of the invention the patient suffering from cancer is prone to, or has previously experienced, an immune-related adverse reaction. In a further preferred aspect of the invention, the patient suffering from cancer also has a pre-existing autoimmune disease.

A sub-clinical dosage of checkpoint inhibitor, such as a PD-1 antagonist, or NK₁ inhibitor is a dosage which provides the same, or substantially the same, effect as a placebo. Thus, a sub-clinical dose typically provides an anticancer effect within in the range of ±20%, preferably ±10%, of that observed with placebo. The amount of drug representing sub-clinical dosage will vary from depending upon the specific active ingredients used, and a suitable dosage can be easily determined by a skilled physician.

Dosage regimens may be adjusted to provide the optimum desired response. For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration may be in single or multiple doses. Multiple doses may be administered via the same or different routes and to the same or different locations. Dosage and frequency may vary depending on the half-life of the drugs in the patient and the duration of treatment desired.

NK₁ inhibitors, such as aprepitant and fosaprepitant, and pharmaceutically acceptable salts thereof, are currently approved for use in treating nausea and vomiting in patients, including those suffering from cancer who may also be suffering from nausea and vomiting as a result of chemotherapy and/or surgery. It is thus preferred in the present invention that the NK₁ inhibitor is not prescribed and/or administered to the patient for the purpose of treating nausea and vomiting, but instead is prescribed and/or administered to the patient for treating cancer.

The present invention is explained in more detail in the following by referring to the Examples, which are not to be construed as limitative.

### EXAMPLES

### Example 1

10⁶ B16F10 mouse melanoma cells were transplanted subcutaneously into syngeneic C57bl/6 male mice. Tumour volume was monitored by direct external measurement with a caliper, and estimated according to the following formula:

V=w²L/2

where V is the tumour volume, w is the width, and L is the length.

When tumours reached 400-500 mm³ (day 7 after transplantation), mice were treated with fosparepitant (IVEMEND®, one daily dose for up to 8 days) and an anti-PD-1 antibody (one dose every other day for up to 8 days). The anti-PD-1 antibody was a Mab anti-mouse PD-1 (CD279) antibody available from Bio X Cell (Clone: RMP1-14, Catalog #: BE0146, Isotype: Rat IgG2a).

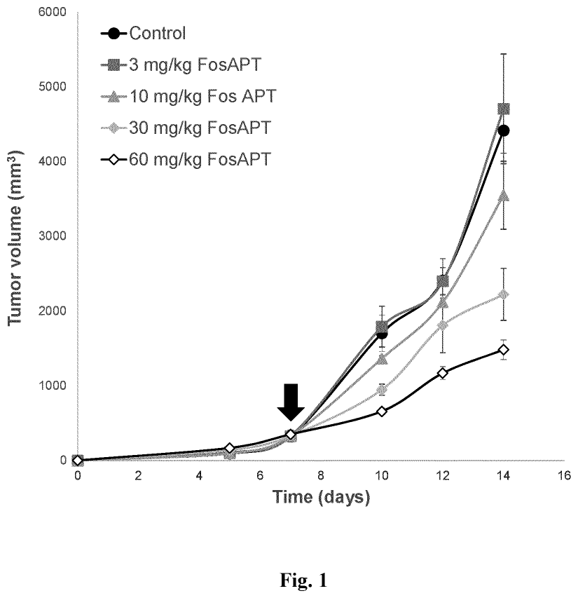

In a first series of experiments, a dose-antitumour effect was obtained for fosaprepitant. Tumour-bearing mice were divided into the following groups:
GROUP 1. CONTROL. Saline, i.p. (n=4)
GROUP 2. 3 mg/kg/day FOSAPREPITANT, i.p. (n=4)
GROUP 3. 10 mg/kg/day FOSAPREPITANT, i.p. (n=4)
GROUP 4. 30 mg/kg/day FOSAPREPITANT, i.p. (n=4)
GROUP 5. 60 mg/kg/day FOSAPREPITANT, i.p. (n=4)
   The results are provided in FIG. 1.

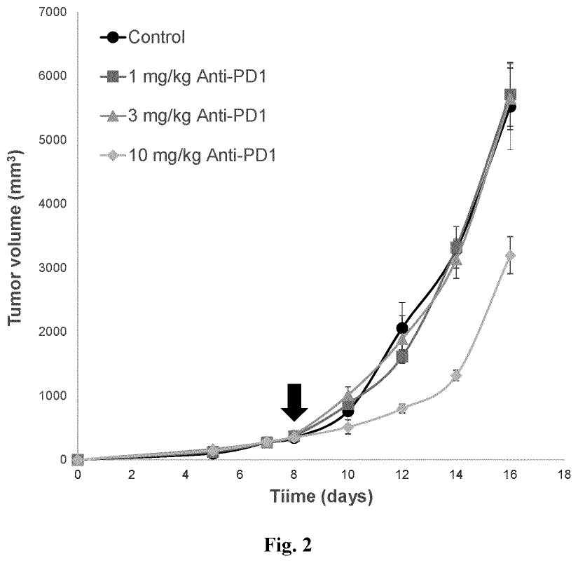

In a second series of experiments, a dose-antitumour effect was obtained for the anti-PD-1 antibody. Tumour-bearing mice were divided into the following groups
GROUP 1. CONTROL. Saline, i.p. (n=4)
GROUP 2. 1 mg/kg/day (in alternate days) ANTI-PD-1, i.p. (n=4)
GROUP 3. 3 mg/kg/day (in alternate days) ANTI-PD-1, i.p. (n=4)
GROUP 4. 10 mg/kg/day (in alternate days) ANTI-PD-1, i.p. (n=4)
   The results are provided in FIG. 2.

In a third series of experiments, the antitumor effect of the combination of fosaprepitant plus anti-PD-1 was tested. For this purpose dosage regimes for both fosparepitant and anti-PD-1 were selected from FIGS. 1 and 2, respectively. In order to be able to substantiate an additive effect of the combination, the dosages of 30 mg/kg/day fosaprepitant and 8 mg/kg/day anti PD-1 antibody (in alternate days) were chosen for single and double therapies. In addition, as an additional control therapy, an isotypic, unspecific immunoglobulin (IgG2a) was also used both in single therapy and in combination with fosaprepitant. The IgG2a used was obtained from Bio X Cell (Clone: 2A3, Catalog #: BE0089, Isotyp: Rat IgG2a).

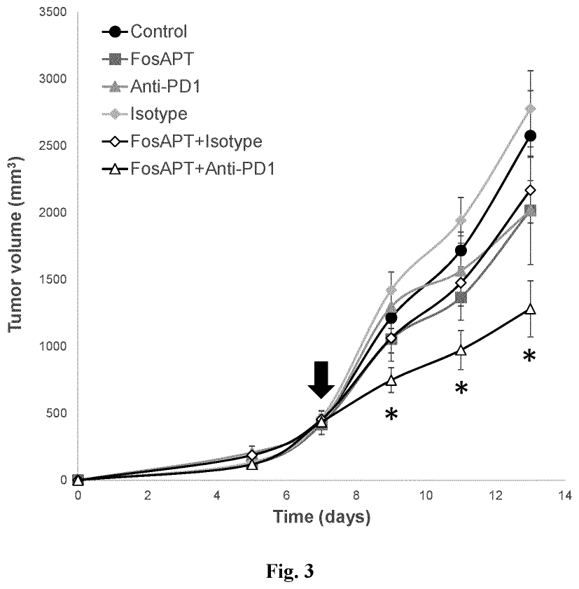

Tumour-bearing mice were divided into the following groups:
GROUP 1. CONTROL. Saline, i.p. (n=6)
GROUP 2. 30 mg/kg/day FOSAPREPITANT, i.p. (n=6)
GROUP 3. 8 mg/kg/day ANTI-PD-1, i.p. (n=6)
GROUP 4. 8 mg/kg/day ISOTYPE IgG, i.p. (n=6)
GROUP 5. 30 mg/kg/day FOSAPREPITANT, i.p. 8 mg/kg/day ISOTYPE IgG, i.p. (n=6)
GROUP 6. 30 mg/kg/day FOSAPREPITANT, i.p. 8 mg/kg/day ANTI-PD-1, i.p. (n=6)
   The results are provided in FIG. 3.

Statistical analysis was performed using the NCSS software. Data were expressed as mean±SEM. A one-way ANOVA analysis was performed. For data conforming to a normal distribution, the Scheffe's correction test was used for multiple comparisons. For data not conforming to a normal distribution, the Kruskal-Wallis Z value test was used for multiple comparison. P<0.05 or Z>1.96 were considered statistically significant. *Z was greater than 1.96 for (a) the combination of fosaprepitant and PD-1 antagonist vs (b) control, (c) the isotype, and (d) the PD-1 antagonist monotherapy.

The results in FIG. 3, in conjunction with those in FIGS. 1 and 2, therefore show that combination of fosaprepitant and a PD-1 antagonist shows enhanced antitumor activity compared with single treatments, as evidenced by lower tumour volume growth.

### Example 2

### Materials & Methods

### Cell Lines

One Lewis Pulmonar Carcinoma cell line: 3LL; and one melanoma cell line: B16F10 (ATCC cell bank; LGC Standards); were used in this study. Both cell lines were maintained in culture in Dulbecco's modified Eagle's medium (DMEM) (Gibco, Thermo Fisher Scientific) supplemented with 10% foetal bovine serum (FBS) (Gibco, Thermo Fisher Scientific), 2 mM L-glutamine (Sigma-Aldrich, Merk) and Peni-Strep (50 U/ml and 50 μl/ml; Gibco, Thermo Fisher Scientific) in a humidified incubator at 37° C. and 5% CO₂.

### Drugs

Fosaprepitant dimeglumine salt (CAS No 265121-04-8) and netupitant (CAS No 290297-26-6) were purchased from Selleck Chemicals. Both were administered intraperitoneally at 30 mg/kg dose, once a day.

Cerenia® (maropitant citrate injectable solution; Zoetis US) was injected intraperitoneally at a dose of 30 mg/kg, once a day.

InVivoMAb Anti-mouse PD-1 (CD279), RMP1-14 clone (ref. BE0146); Rat IgG2a, k isotype (ref BE0089);

InVivoMAb Anti-mouse CTLA-4 (CD152), UC10-4F10-11 clone (ref. BE0032) band its corresponding isotype polyclonal armenian Hamster IgG (ref. BE0091) were purchased from Bio X Cell.

Anti-mouse PD-1 and its isotype were used at 8 mg/kg dose; CTLA-4 and its isotype at 5 mg/kg dose. All were administrated every two days, intraperitoneally.

### Xenograft Mouse Model

3LL cells (0.6×10⁶ per mouse) or B16F10 cells (1×10⁶ per mouse) were subcutaneously injected into the right flank of C57BL/6J mice (Charles River) with matrigel (Matrigel Membrane Matrix, Corning) at 50% v/v. Tumor growth was evaluated every 2/3 days. Animal vital parameters were monitored daily. When the tumors reached an average volume of 100-150 mm³, mice were randomized into cohorts of 6 animals each and treatments were administered for 7-9 days. Groups and regimens for each experiment are described in table 1. Animals of the control group were dosed with equal volume of vehicle. Animals were euthanized according to institutional guidelines and tumor samples were excised. Tumors were snap-frozen in OCT medium (Sakura Tissue Tek) or fixed in PFA and embedded in paraffin. Several internal organs were also excised and fixed in PFA for future studies.

TABLE 1
Mice groups and regimens per experiment.

| | | | |
|---|---|---|---|
| | | Route of | |
| Group | Dose | administration | Regimen |

Fosaprepitant/netupitant in combination with anti-PD-1. 3LL cell line

| | | | |
|---|---|---|---|
| Control | Same volume as | Intraperitoneal | Once a day |
| | treatments | injection | |
| Isotype | 8 mg/kg | Intraperitoneal | Every other day |
| | | injection | |
| Anti PD-1 | 8 mg/kg | Intraperitoneal | Every other day |
| | | injection | |
| Fosaprepitant | 30 mg/kg | Intraperitoneal | Once a day |
| | | injection | |
| Fosaprepitant- | 30 mg/kg-8 | Intraperitoneal | Once a day-Every |
| isotype | mg/kg | injection | other day |
| Fosaprepitant-anti | 30 mg/kg-8 | Intraperitoneal | Once a day-Every |
| PD-1 | mg/kg | injection | other day |
| Netupitant | 30 mg/kg | Intraperitoneal | Once a day |
| | | injection | |
| Netupitant-isotype | 30 mg/kg-8 | Intraperitoneal | Once a day-Every |
| | mg/kg | injection | other day |
| Netupitant-anti PD-1 | 30 mg/kg-8 | Intraperitoneal | Once a day-Every |
| | mg/kg | injection | other day |

Maropitant in combination with anti-CTLA-4. B16F10 cell line

| | | | |
|---|---|---|---|
| Control | Same volume as | Intraperitoneal | Once a day |
| | treatments | injection | |
| Anti CTLA-4 | 5 mg/kg | Intraperitoneal | Every other day |
| | | injection | |
| Maropitant | 30 mg/kg | Intraperitoneal | Once a day |
| | | injection | |
| Maropitant-anti | 30 mg/kg-5 | Intraperitoneal | Once a day-Every |
| CTLA-4 | mg/kg | injection | other day |

Results—Example 2a: Fosaprepitant/Netupitant Combination with Anti-PD-1

In order to evaluate the effect of Fosaprepitant+anti PD-1 and Netupitant+anti PD-1 combinations in vivo, 3LL tumor-bearing mice were randomly assigned into nine treatment groups as detailed in Table 1 (n=6 mice per group) to receive either fosaprepitant 30 mg/kg, netupitant 30 mg/kg, anti-PD-1 8 mg/kg or its isotype, the combination of both agents or the equivalent volume of vehicle. Tumor growth was recorded as detailed in Materials and Methods.

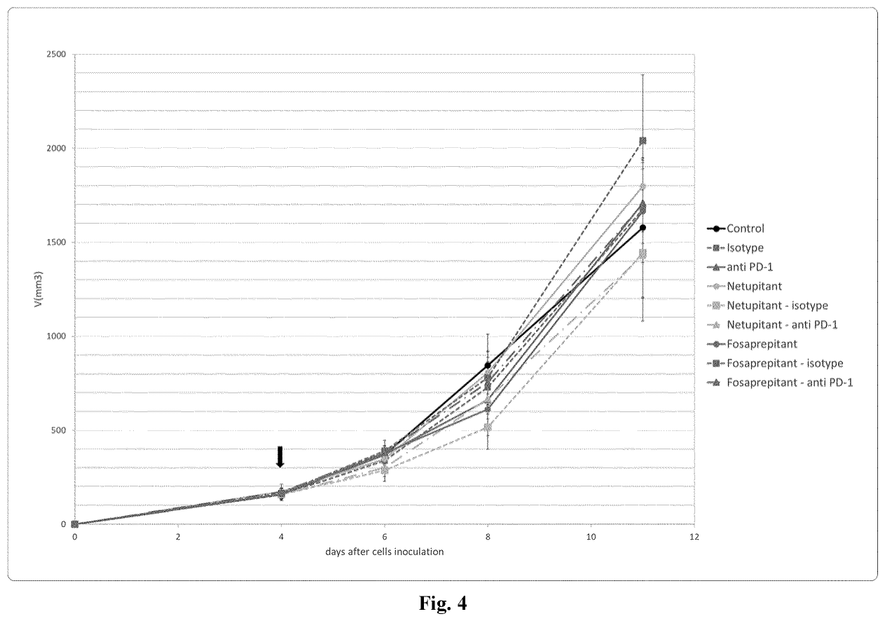

In particular, 0.6×106 3LL cells were subcutaneously inoculated into the right flank of C57BL/6J mice. Tumor-bearing animals (n=6 mice per group) received intraperitoneal injection of 30 mg/kg Fosaprepitant, 30 mg/kg Netupitant, 8 mg/kg anti PD-1, 8 mg/kg anti PD-1 isotype, combinations between them, or an equal volume of vehicle, for 7 days. Tumor volumes were measured each 2-3 days with external calipers. The results are depicted in FIG. 4. All tested treatments failed to inhibit tumor growth of pulmonary carcinoma tumors. No statistical significant differences were observed. The arrow represents the first day of treatment (day 4 after cells inoculation).

Results—Example 2h: Fosaprepitant/Maropitant Combination with Anti-CTLA-4

In order to evaluate the effect of Maropitant and anti CTLA-4 combination in vivo, B16F10 tumor-bearing mice were randomly assigned into four treatment groups as detailed in table 1 (n=6 mice per group) to receive either Maropitant 30 mg/kg, anti CTLA-4 5 mg/kg, the combination of both agents or the equivalent volume of vehicle. Tumor growth was recorded as detailed in Materials and Methods.

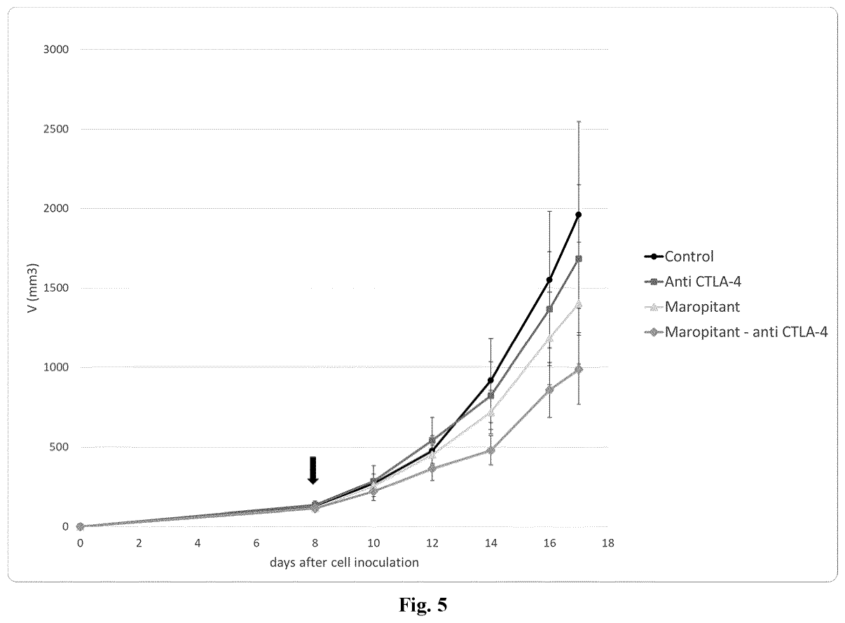

In particular, 1×10⁶ B16F10 cells were subcutaneously inoculated into the right flank of C57BL/6J mice. Tumor-bearing animals (n=6 mice per group) received intraperitoneal injection of 30 mg/kg maropitant, 5 mg/kg anti-CTLA-4, its combination, or an equal volume of vehicle for 9 days. Tumor volumes were measured each 2-3 days with external calipers. The results are depicted in FIG. 5. The arrow represents the first day of treatment (day 8 after cells inoculation).

Maropitant as a single agent allowed a 28% reduction in tumor growth after 9 days of treatment and anti CTLA-4 treatment showed a 14% inhibition of tumor growth when compared to vehicle group. The combination of Maropitant and anti CTLA-4 allowed a reduction up to 50% in tumor growth, which is a greater than additive effect compared to the monotherapies.

1. A pharmaceutical composition which comprises:
(a) a NK₁ inhibitor; and
(b) a checkpoint inhibitor;
for use in treating cancer.

2. The pharmaceutical composition for use according to claim 1, wherein the NK₁ inhibitor is aprepitant, fosaprepitant, netupitant, maropitant, vestipitant, casopitant, vofopitant, ezlopitant or lanepitant, or a pharmaceutically acceptable salt thereof.

3. The pharmaceutical composition for use according to claim 2, wherein the NK₁ inhibitor is aprepitant or fosaprepitant, or pharmaceutically acceptable salt thereof.

4. The pharmaceutical composition for use according to claim 3, wherein the NK₁ inhibitor is aprepitant.

5. The pharmaceutical composition for use according to claim 3, wherein the NK₁ inhibitor is fosaprepitant dimeglumine.

6. The pharmaceutical composition for use according to anyone of the preceding claims, wherein the checkpoint inhibitor blocks or inhibits one or more immune system checkpoints selected from:
a. The interaction between PD-1 and PDL-1 and/or PD-1 and PDL-2;
b. The interaction between CTLA-4 and CD86 and/or CTLA-4 and CD80;
c. The interaction between B7-H3 and/or B7-H4 and their respective ligands;
d. The interaction between HVEM and BTLA;
e. The interaction between GALS and TIM3;
f. The interaction between MHC class I or II and LAG3; and
g. The interaction between MHC class I or II and KIR

7. The pharmaceutical composition for use according to claim 6, wherein the checkpoint inhibitor is a PD-1 antagonist.

8. The pharmaceutical composition for use according to claim 7, wherein the PD-1 antagonist is nivolumab, pembrolizumab or pidilizumab.

9. The pharmaceutical composition for use according to claim 6, wherein the checkpoint inhibitor is a CTLA-4 antagonist.

10. The pharmaceutical composition for use according to claim 9, wherein the CTLA-4 antagonist is ipilumumab or tremelimumab.

11. The pharmaceutical composition for use according to any one of the preceding claims, wherein the cancer is melanoma, lung cancer, renal cell carcinoma, lymphoma, squamous cell cancer, or urothelial carcinoma, preferably melanoma, renal cell carcinoma, lymphoma, squamous cell cancer, or urothelial carcinoma, most preferably melanoma.

12. The pharmaceutical composition for use according to any one of the preceding claims, wherein at least 1% of the cells in a tumour of the cancer express PD-1 and/or PD1-1, as determined by immunohistochemistry.

13. A NK₁ inhibitor as defined in any one of claims 1 to 5, for use in treating cancer as defined in claim 1, 11 or 12. by co-administration with a checkpoint inhibitor as defined in any one of claims 1 and 6 to 10.

14. A checkpoint inhibitor as defined in any one of claims 1 and 6 to 10, for use in treating cancer as defined in claim 1, 11 or 12. by co-administration with a NK₁ inhibitor as defined in any one of claims 1 to 5.

15. A method of treating a patient suffering from cancer as defined in claim 1, 11 or 12, which method comprises co-administering to said patient (a) a NK₁ inhibitor as defined in any one of claims 1 to 5, and (b) a checkpoint inhibitor as defined in any one of claims 1 and 6 to 10.

16. A product comprising (a) a NK₁ inhibitor as defined in any one of claims 1 to 5, and (b) a checkpoint inhibitor as defined in any one of claims 1 and 6 to 10, as a combined preparation for simultaneous, concurrent, separate or sequential use in the treatment of a patient suffering from cancer as defined in claim 1, 11 or 12.

17. Use of a NK₁ inhibitor as defined in any one of claims 1 to 5 in the manufacture of a medicament for the treatment of cancer as defined in claim 1, 11 or 12, by co-administration with a checkpoint inhibitor as defined in any one of claims 1 and 6 to 10.

18. Use of a checkpoint inhibitor as defined in any one of claims 1 and 6 to 10 in the manufacture of a medicament for the treatment of cancer as defined in claim 1, 11 or 12, by co-administration with a NK₁ inhibitor as defined in any one of claims 1 to 5.

19. A pharmaceutical composition which comprises:
(a) a NK₁ inhibitor as defined in any one of claims 1 to 5; and
(b) a checkpoint inhibitor as defined in any one of claims 1 and 6 to 10.

20. A kit which comprises:
(a) a pharmaceutical composition comprising a NK₁ inhibitor as defined in any one of claims 1 to 5; and
(b) a pharmaceutical composition comprising a checkpoint inhibitor as defined in any one of claims 1 and 6 to 10.
